(19) United States
(12) Patent Application Publication
(10) Pub. No.: US 2021/0193171 A1
(43) Pub. Date: Jun. 24, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Akira ICHIBOSHI, Kanagawa (JP); Kazunari KOMATSUZAKI, Kanagawa (JP); Ryota MIZUTANI, Kanagawa (JP); Shingo UCHIHASHI, Kanagawa (JP)

(73) Assignee: FUJI XEROX CO., LTD., Tokyo (JP)

(21) Appl. No.: 16/889,060

(22) Filed: Jun. 1, 2020

(30) Foreign Application Priority Data
Dec. 23, 2019 (JP) 2019-231254

(51) Int. Cl. G10L 25/63; G10L 25/66; A61B 5/00

(52) U.S. Cl. CPC G10L 25/63; A61B 5/7296; G10L 25/66

(57) ABSTRACT

An information processing apparatus includes a processor. The processor is configured to determine a level of load on a user in accordance with communication-related information of the user and biologically-related information of the user, identify a factor that applies the load on the user in accordance with the level, and output the identified factor.

### CROSS-REFERENCE TO RELATED APPLICATIONS

This application is based on and claims priority under 35 USC 119 from Japanese Patent Application No. 2019-231254 filed Dec. 23, 2019.

### BACKGROUND

### (i) Technical Field

The present disclosure relates to information processing apparatuses and non-transitory computer readable media.

### (ii) Related Art

Japanese Unexamined Patent Application Publication No. 2016-209404 proposes an example of a stress detection system in the related art that is capable of detecting when and where the stress level increases and identifying the cause of the increase in the stress level.

The stress detection system described in Japanese Unexamined Patent Application Publication No. 2016-209404 includes a characteristic-value detection sensor that continuously detects a characteristic value of a subject, a condition recorder that continuously records the condition surrounding the subject, a position detector that continuously detects the position of the subject, a timekeeper that continuously measures time, a stress evaluator that continuously determines whether or not a difference between the characteristic value detected by the characteristic-value detection sensor and a reference value has exceeded a predetermined threshold value, and a stress-data storage unit. When the stress evaluator determines that the difference between the characteristic value and the reference value has exceeded the predetermined threshold value, the stress-data storage unit stores the time point at which the difference has exceeded the predetermined threshold value, the condition recorded by the condition recorder in a predetermined time period including the time point at which the difference has exceeded the predetermined threshold value, and the position recorded by the position detector in the predetermined time period in association with one another.

### SUMMARY

A method that uses the voice of a user or an image of the surrounding environment for identifying the factor that applies a load on the user may possibly lead to problems in terms of information security and privacy protection.

Aspects of non-limiting embodiments of the present disclosure relate to an information processing apparatus and a non-transitory computer readable medium that are capable of identifying the factor that applies a load on a user without taking into account the content of a speech.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including a processor. The processor is configured to determine a level of load on a user in accordance with communication-related information of the user and biologically-related information of the user, identify a factor that applies the load on the user in accordance with the level, and output the identified factor.

### BRIEF DESCRIPTION OF THE DRAWINGS

Exemplary embodiments of the present disclosure will be described in detail based on the following figures, wherein:

FIG. 1 is a block diagram illustrating an example of the configuration of an information processing system according to a first exemplary embodiment of the present disclosure;

FIG. 2 is a block diagram illustrating an example of a control system of an information processing apparatus according to the first exemplary embodiment;

FIGS. 3A to 3C illustrate examples of a communication information table;

FIG. 4 is a flowchart illustrating an example of the operation of the information processing apparatus according to the first exemplary embodiment;

FIG. 5 is a flowchart illustrating an example of a process for checking the stress level and identifying the factor of stress in the flowchart shown in FIG. 4;

FIG. 6 is a block diagram illustrating an example of a control system of an information processing apparatus according a second exemplary embodiment of the present disclosure;

FIG. 7 is a flowchart illustrating a process for checking the stress level and identifying the factor of stress in accordance with the second exemplary embodiment;

FIG. 8 is a block diagram illustrating an example of a control system of an information processing apparatus according to a third exemplary embodiment of the present disclosure;

FIG. 9 illustrates an example of abnormality levels;

FIG. 10 is a block diagram illustrating an example of a control system of an information processing apparatus according to a fourth exemplary embodiment of the present disclosure; and

FIG. 11 illustrates an example of how the factor of stress is distinguished.

### DETAILED DESCRIPTION

Exemplary embodiments of the present disclosure will be described below with reference to the drawings. In the drawings, components substantially having identical functions are given the same reference signs, and redundant descriptions thereof are omitted.

### First Exemplary Embodiment

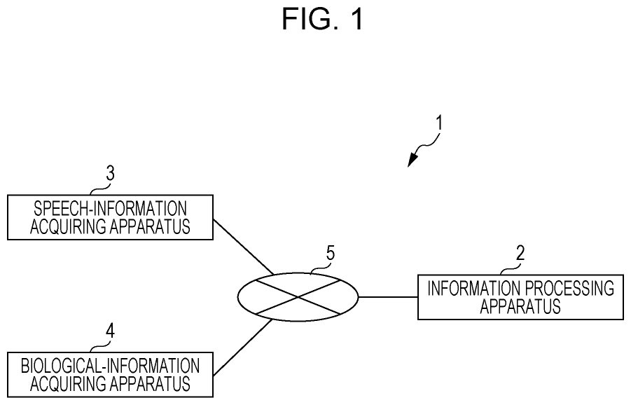

FIG. 1 illustrates an example of the configuration of an information processing system according to a first exemplary embodiment of the present disclosure. An information processing system 1 is applied to a place or an area (also referred to as “activity area” hereinafter) where a user is active. Examples of the activity area include a room (including a rental office and a shared office, and also referred to as “office” hereinafter), a workplace, such as a factory, and a learning place, such as a school or a classroom. FIG. 1 illustrates a case where the information processing system 1 is applied to an office.

As shown in FIG. 1, the information processing system 1 includes an information processing apparatus 2, a speech-information acquiring apparatus 3, a biological-information acquiring apparatus 4, and a network 5 that connects the information processing apparatus 2, the speech-information acquiring apparatus 3, and the biological-information acquiring apparatus 4 in a communicable manner.

The information processing apparatus 2 may be, for example, a personal computer or a portable information terminal, such as a tablet terminal or a multifunction portable telephone (smartphone). The information processing apparatus 2 will be described in detail later.

The speech-information acquiring apparatus 3 acquires information indicating the position of the user (also referred to as “positional information” hereinafter) and speech-related information (also referred to as “speech information” hereinafter). The speech-information acquiring apparatus 3 may be, for example, a detector that contains a camera and a directional microphone.

The biological-information acquiring apparatus 4 measures, for example, biologically-related information of the user when the user is active in the activity area (also referred to as “biological information” hereinafter). The biological-information acquiring apparatus 4 may measure the biological information not only when the user is active but also when, for example, the user is in an inactive state, such as when the user is lying down, napping, or sleeping.

The biological information is released from a biological body and may include any of the following examples:

a. information indicating a body motion (e.g., acceleration caused by a body motion, a pattern indicating a behavior, and so on);

b. an amount of activity (e.g., the number of steps taken, consumed calories, and so on); and

c. vital information (e.g., the heart rate, the pulse wave, the pulse rate, the respiration rate, the body temperature, the blood pressure, and so on).

The biological-information acquiring apparatus 4 is desirably of a wearable type worn on the body of the user. Examples of the wearable type include a wristband type worn on a wrist, a ring type worn on a finger, a belt type worn on the waist, a shirt type that comes into contact with, for example, the left and right arms, the shoulders, the chest, and the back, an eyeglasses type or a goggle type worn on the head, an earphone type worn on an ear, and an attachable type attached to a part of the body.

In this exemplary embodiment, the biological-information acquiring apparatus 4 used is of a wristband type, but may alternatively be of another type or a combination of multiple types. Moreover, the biological-information acquiring apparatus 4 does not necessarily have to be worn on the body. For example, the biological-information acquiring apparatus 4 may be a camera having a function for measuring the heart rate by capturing the absorption of light by hemoglobin.

The network 5 is a communication network, such as a local area network (LAN), a wide area network (WAN), the Internet, or an intranet, and may be a wired network or a wireless network.

Configuration of Information Processing Apparatus 2

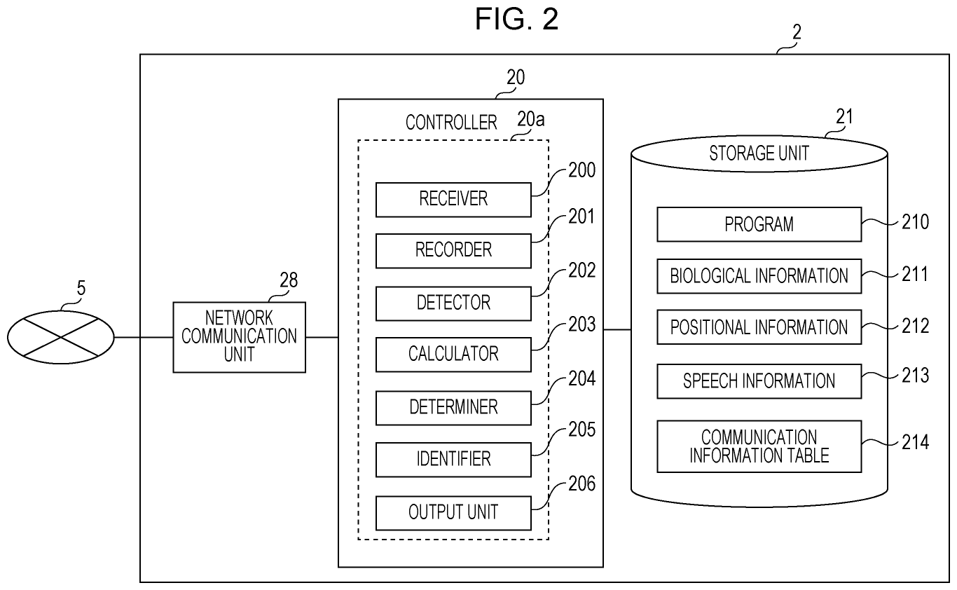

FIG. 2 is a block diagram illustrating an example of a control system of the information processing apparatus 2. As shown in FIG. 2, the information processing apparatus 2 includes a controller 20 that controls each component, a storage unit 21 that stores various types of data, and a network communication unit 28 that communicates with the speech-information acquiring apparatus 3 and the biological-information acquiring apparatus 4 via the network 5.

The controller 20 is constituted of, for example, a processor 20a, such as a central processing unit (CPU), and an interface. The processor 20a operates in accordance with a program 210 stored in the storage unit 21 so as to function as, for example, a receiver 200, a recorder 201, a detector 202, a calculator 203, a determiner 204, an identifier 205, and an output unit 206. The components 200 to 206 will be described in detail later.

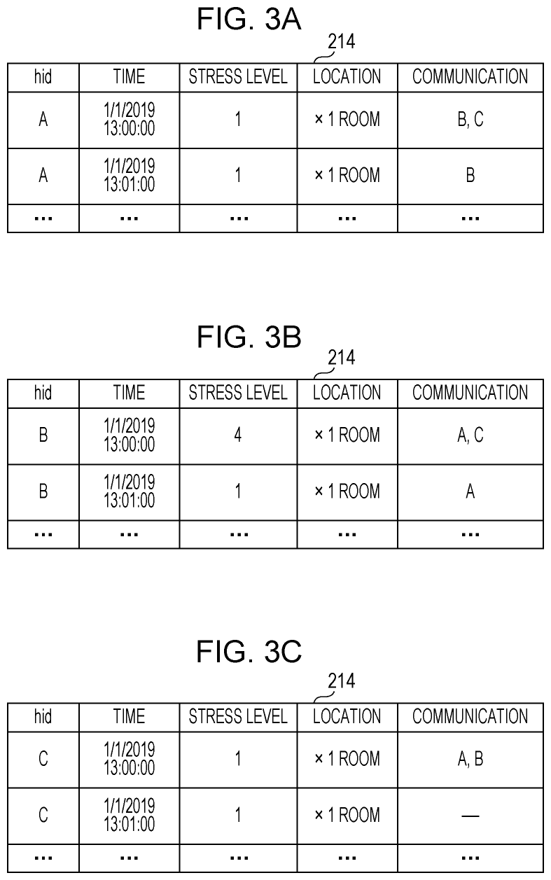

The storage unit 21 is constituted of, for example, a read-only memory (ROM), a random access memory (RAM), and a hard disk, and stores therein various types of data, such as the program 210, biological information 211, positional information 212, speech information 213, and a communication information table 214 (see FIGS. 3A to 3C).

The biological information 211, the positional information 212, and the speech information 213 are acquired from the speech-information acquiring apparatus 3 and the biological-information acquiring apparatus 4, and are stored in association with information for identifying a user, such as a user ID. The positional information 212 and the speech information 213 are examples of communication information.

In the biological information 211, the positional information 212, and the speech information 213, past information may be further recorded as history information, in addition to the current user-related information acquired from the speech-information acquiring apparatus 3 and the biological-information acquiring apparatus 4. The communication information table 214 will be described in detail later.

The network communication unit 28 is realized by, for example, a network interface card (NIC), and exchanges various types of information and signals with the speech-information acquiring apparatus 3 and the biological-information acquiring apparatus 4 via the network 5.

The receiver 200 receives various types of information and signals transmitted to the information processing apparatus 2 from an external apparatus. In detail, the receiver 200 receives the biological information 211, the positional information 212, and the speech information 213 transmitted from the speech-information acquiring apparatus 3 and the biological-information acquiring apparatus 4.

The recorder 201 records the various types of information received by the receiver 200 into the biological information 211, the positional information 212, and the speech information 213 in the storage unit 21. Furthermore, the recorder 201 records a communication status and a change in the communication status, obtained from the various types of information received by the receiver 200, onto the communication information table 214 in the storage unit 21.

The detector 202 detects the number of users involved in communication (also referred to as “number of people involved” hereinafter) in accordance with the positional information 212 and the speech information 213.

A “user involved in communication” is not necessarily limited to a user who is currently speaking out a certain kind of information, and may include a user acting as a dedicated listener, a user conversing with a currently-speaking user and waiting for the currently-speaking user to end his/her speech (when the users are taking turns in speaking), and so on.

For each user, the calculator 203 calculates the level of load (also referred to as “stress” hereinafter) on the user in accordance with the biological information 211. The level of load may also be referred to as “stress level” hereinafter. The term “stress” refers to a load that may affect the internal state, such as the mental state or the psychological state.

For example, the stress level may be calculated in accordance with a preliminarily-defined process by using information, such as vital information, as an input value. In this exemplary embodiment, the stress level is expressed with the integers “1”, “2”, “3”, and “4”, such that a lower value indicates a lower stress level.

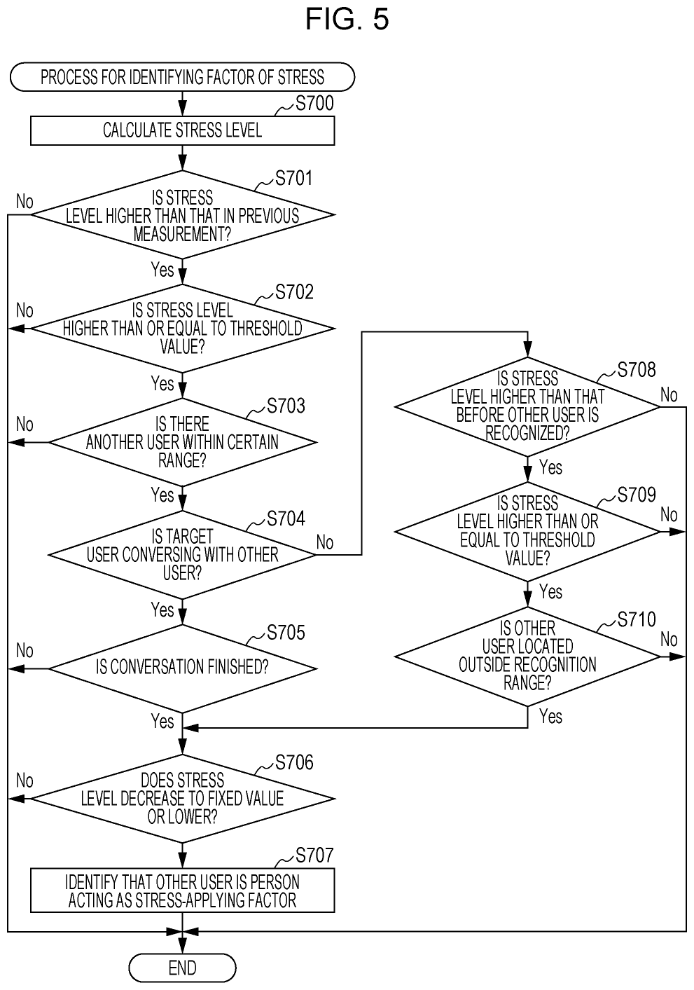

The determiner 204 determines whether or not various conditions are satisfied. The details of contents to be determined by the identifier 205 will be described with reference to a flowchart shown in FIG. 5.

The identifier 205 identifies the factor that applies stress on the user. In detail, the identifier 205 identifies, as a “factor”, a person applying stress on the user by a remark or a tangible or intangible pressure (including silence and neglect), an event that is causing a stressful situation or environment for the user, and so on. For example, the identifier 205 identifies the factor in accordance with how the stress level changes before and after a speech by the user. This person is an example of a load-applying person.

The output unit 206 outputs information based on the factor identified by the identifier 205. For example, the output unit 206 may notify the person identified as the “factor” by the identifier 205 that the person is applying stress on the user. Alternatively, the output unit 206 may output information related to the person or event identified as the “factor” by the identifier 205 in the form of a report, or may output the information as data to another management apparatus (not shown).

Configuration of Table

FIGS. 3A to 3C illustrate examples of the communication information table 214. The communication information table 214 contains a record of information related to a change in the communication status of each user as a measurement target. The following description relates to an example of communication among three users, namely, a user A (which may simply be referred to as “A” hereinafter), a user B (which may simply be referred to as “B” hereinafter), and a user C (which may simply be referred to as “C” hereinafter).

FIG. 3A illustrates an example of the communication information table 214 related to the user A, FIG. 3B illustrates an example of the communication information table 214 related to the user B, and FIG. 3C illustrates an example of the communication information table 214 related to the user C.

As shown in each of FIGS. 3A to 3C, the communication information table 214 is provided with a “hid” field, a “time” field, a “stress level” field, a “location” field, and a “communication” field. In the “hid” field, information for identifying a user as a subject is recorded.

In the “time” field, the time at which a certain change has occurred in the communication status is recorded. In this case, for example, the time at which a change has occurred in the configuration of the user involved in communication is recorded in the “time” field.

In the “stress level” field, a value indicating the stress level of the user recorded in the “hid” field is recorded. In the “location” field, information indicating the current activity area of the user recorded in the “hid” field is recorded. In the “communication” field, information for identifying a user or users currently conversing with the user recorded in the “hid” field is recorded.

An example will be described. As shown in FIG. 3A, the user A is conversing with the user B and the user C in an “X1 room” at “13:00:00 on Jan. 1, 2019” (data in the first row). In this case, the recorded stress level of the user A is “1”. Then, assuming that the user C leaves the conversation after one minute so that the user A and the user B are conversing with each other, “13:01:00 on Jan. 1, 2019” (data in the second row) is recorded in the “time” field, and “B” is recorded in the “communication” field.

From the viewpoint of the user B, as shown in FIG. 3B, the recorded information indicates that the user B is conversing with the user A and the user C in the “X1 room” at “13:00:00 on Jan. 1, 2019” (data in the first row). Since the user C leaves the conversation after one minute so that the only person conversing with the user B is the user A, “A” alone is recorded in the “communication” field at “13:01:00 on Jan. 1, 2019” (data in the second row). Meanwhile, the stress level of the user B has decreased from “4” to “1”.

Specifically, the stress level of the user B has decreased from “4” to “1” as a result of the user C leaving the conversation. In such a case, the identifier 205 may identify that the user C is the factor applying stress on the user B. A detailed description of the communication information table 214 for the user C (FIG. 3C) will be omitted.

Operation According to First Exemplary Embodiment

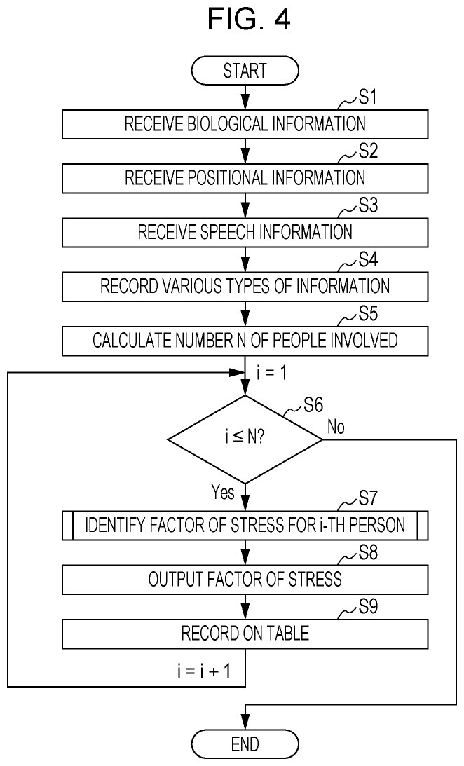

FIG. 4 is a flowchart illustrating an example of the operation of the information processing apparatus 2. The speech-information acquiring apparatus 3 and the biological-information acquiring apparatus 4 acquire the biological information 211, the positional information 212, and the speech information 213 of a user, and transmits these various types of information to the information processing apparatus 2. The receiver 200 receives the biological information 211, the positional information 212, and the speech information 213 of the user transmitted from the apparatuses in step S1, step S2, and step S3.

In step S4, the recorder 201 records the various types of information received by the receiver 200 into the biological information 211, the positional information 212, and the speech information 213 in the storage unit 21.

Then, in step S5, the detector 202 detects users involved in communication in accordance with the speech information 213 received by the receiver 200, and calculates the number of people involved. The number of people involved is defined as N (N being a natural number of 1, 2, 3, or so on).

Subsequently, for each user (i.e., an “i-th user”, i=1, 2, . . . , N) detected by the detector 202 (YES in step S6), the calculator 203, the determiner 204, and the identifier 205 execute a process (also referred to as “stress checking process” hereinafter) in step S7 for checking the stress level and identifying the factor of the stress. The process for checking the stress level and identifying the factor of the stress will be described in detail later with reference to FIG. 5.

In step S8, the output unit 206 outputs the identified factor of the stress. Furthermore, in step S9, the recorder 201 records the stress level, the communication status, and a change in the communication status onto the communication information table 214.

FIG. 5 is a flowchart illustrating an example of the process for checking the stress level and identifying the factor of the stress in the flowchart shown in FIG. 4. In step 5700, the calculator 203 calculates the stress level of the user (i.e., the “i-th user” serving as a target for the process for checking the stress level and identifying the factor of the stress in FIG. 4 and also referred to as “target user” or “user of interest” hereafter for distinguishing the user from other users).

The identifier 205 identifies the factor of the stress in accordance with how the stress level changes before and after a speech by the target user. In detail, the determiner 204 first determines in step S701 whether the calculated stress level is higher or lower than that in the previous measurement. If the calculated stress level is higher than that before the target user starts speaking (YES in step S701), the determiner 204 determines in step S702 whether or not the stress level of the target user is higher than or equal to a predetermined threshold value.

Subsequently, if the stress level of the target user is higher than or equal to the predetermined threshold value (YES in step S702), the determiner 204 refers to the positional information 212 stored in the storage unit 21 so as to determine in step S703 whether or not there is another user within a certain range of the target user.

If there is another user within the certain range of the target user (YES in step S703), the determiner 204 determines in step S704 whether or not the target user is conversing with the other user.

If the target user is conversing with the other user (YES in step S704), when the target user has finished conversing with the other user (YES in step S705), the determiner 204 determines in step S706 whether or not the stress level of the target user decreases to a predetermined fixed value or lower before and after the conversation with the other user.

The “predetermined fixed value” corresponds to, for example, a normal stress level for the target user. Furthermore, the “normal stress level” may be, for example, an average value of past stress levels of the target user over a certain period of time.

If the stress level of the target user does not decrease to the predetermined fixed value or lower before and after the conversation with the other user (YES in step S706), the identifier 205 identifies that the other user is a person acting as the factor applying stress on the target user in step S707.

Furthermore, if there is another user within the certain range of the target user (YES in step S703) but the target user is not conversing with the other user (NO in step S704), the determiner 204 determines in step S708 whether or not the stress level of the target user is higher than that before the target user recognizes the other user. The target user may recognize the other user based on determination that the target user and the other user are within a predetermined range from the positional information of the target user and the other user.

If the stress level of the target user is higher than that before the target user recognizes the other user (YES in step S708), the determiner 204 determines in step S709 whether or not the stress level of the target user is higher than or equal to a predetermined threshold value. The “threshold value” in step S709 does not have to be the same value as the “threshold value” in step S702. In the description below, similar explanations may sometimes be omitted.

If the stress level of the target user is higher than or equal to the predetermined threshold value (YES in step S709), the determiner 204 refers to the positional information 212 so as to determine in step S710 whether the other user is located outside a range recognized by the target user (also referred to as “recognition range” hereinafter) or inside the recognition range.

If the other user is located outside the recognition range of the target user (YES in step S710), step S706 and step S707 described above are executed. Specifically, the determiner 204 determines in step S706 whether or not the stress level of the target user decreases to the predetermined fixed value or lower, and the identifier 205 identifies in step S707 that the other user is a person acting as the factor applying stress on the target user.

### Second Exemplary Embodiment

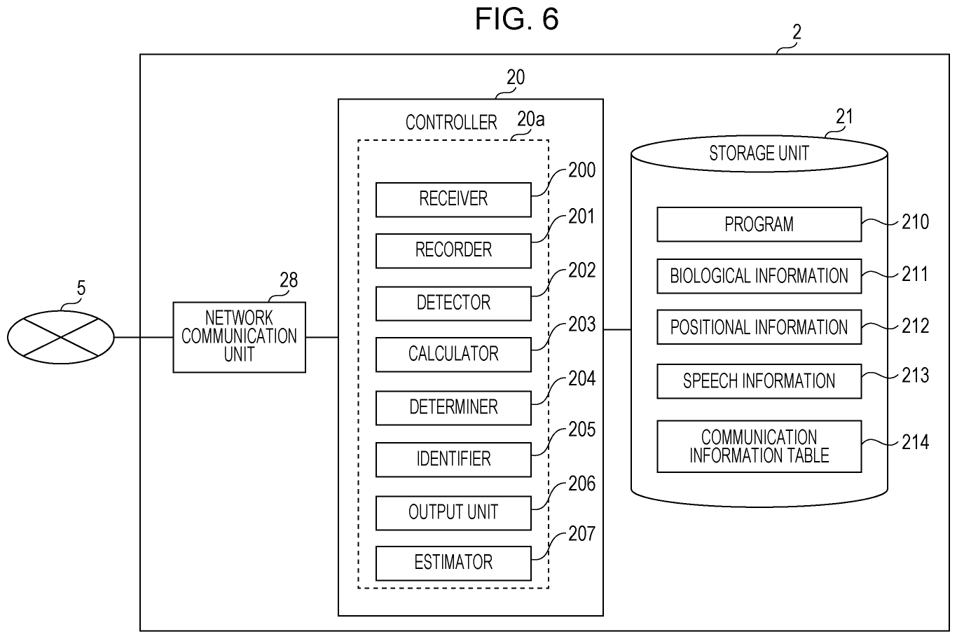

FIG. 6 is a block diagram illustrating an example of the configuration of an information processing apparatus 2 according a second exemplary embodiment of the present disclosure. The controller 20 in the information processing apparatus 2 according to the second exemplary embodiment is different from that in the information processing apparatus 2 according to the first exemplary embodiment in that the controller 20 further includes an estimator 207. Components having configurations and functions identical to those in the first exemplary embodiment are given the same reference signs, and detailed descriptions thereof will be omitted. Moreover, the following description focuses on differences from the information processing apparatus 2 according to the first exemplary embodiment.

The processor 20a further functions as the estimator 207 by operating in accordance with the program 210 stored in the storage unit 21. The estimator 207 estimates the stress level of a target user. In detail, the estimator 207 estimates the stress level of the target user by estimating a score given in accordance with the effect of stress applied on the target user by another user communicating with the target user.

More specifically, the estimator 207 estimates the stress level of the target user in accordance with a score obtained by adding a score by the other user satisfying a certain condition (also referred to as “first condition” hereinafter) against the target user and subtracting a score by the other user satisfying another certain condition (also referred to as “second condition” hereinafter) against the target user.

The “first condition” corresponds to a case where, for example, the stress level is higher than that before the communication and is also higher than or equal to a predetermined threshold value. The “second condition” corresponds to a case where, for example, the stress level is lower than the predetermined threshold value before and after the communication.

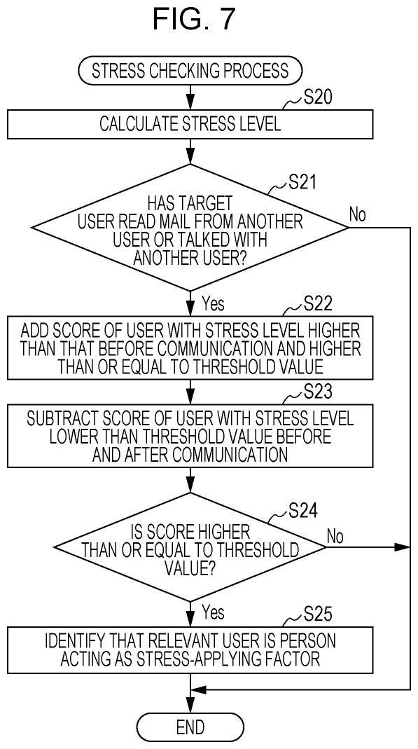

FIG. 7 is a flowchart illustrating the process for checking the stress level and identifying the factor of stress in accordance with the second exemplary embodiment. Communication information used in the process for checking the stress level and identifying the factor of the stress of the target user is not necessarily limited to the positional information 212 and the speech information 213, as in the first exemplary embodiment, and may be, for example, information indicating whether or not a mail transmitted from another user has been read. The action of reading or not reading a mail is an example of communication information.

As shown in FIG. 6, in step S20, the calculator 203 first calculates the stress level of the target user. Then, in step S21, the determiner 204 determines whether or not the target user has read a mail from another user or has talked with another user.

If the target user has read a mail from another user or has talked with another user (YES in step S21), the estimator 207 adds the score of the other user satisfying the aforementioned first condition, that is, the score of the other user with a stress level higher than that before the communication and higher than or equal to the predetermined threshold value, in step S22.

Then, in step S23, the estimator 207 subtracts the score of the other user satisfying the aforementioned second condition, that is, the score of the other user with a stress level lower than the predetermined threshold value before and after the communication.

In step S24, the determiner 204 determines whether or not the score estimated by the estimator 207 is higher than or equal to a predetermined threshold value. If the score is higher than or equal to the predetermined threshold value (YES in step S24), the identifier 205 identifies in step S25 that the relevant user is a person acting as the stress-applying factor.

### Third Exemplary Embodiment

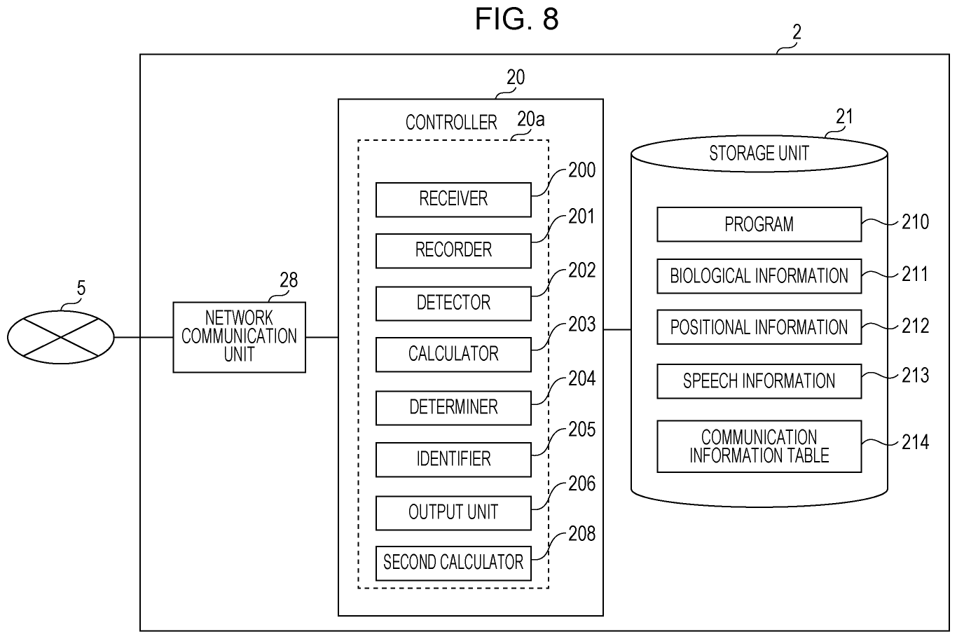

FIG. 8 is a block diagram illustrating an example of the configuration of an information processing apparatus 2 according to a third exemplary embodiment of the present disclosure. In a case where the calculator 203 described in the first exemplary embodiment is defined as a first calculator 203, the controller 20 in the information processing apparatus 2 according to the third exemplary embodiment is different from that in the information processing apparatus 2 according to the first exemplary embodiment in that the controller 20 further includes a second calculator 208. Components having configurations and functions identical to those in the first exemplary embodiment are given the same reference signs, and detailed descriptions thereof will be omitted. Moreover, the following description focuses on differences from the information processing apparatus 2 according to the first exemplary embodiment.

The processor 20a further functions as the second calculator 208 by operating in accordance with the program 210 stored in the storage unit 21. The second calculator 208 calculates the stress level (also referred to as “abnormality level” hereinafter) applied on a target user by another user.

In detail, the second calculator 208 calculates, as an abnormality level, an evaluation value obtained by temporally averaging out stress levels of the target user. The abnormality level is an example of an evaluation value obtained by evaluating stress levels in a time-series fashion. In a conceivable case where the stress level of the target user is measured 10 times every three minutes, it is assumed that the evaluation value is obtained by measuring the stress level 10 times in a time-series fashion so that “1, 1, 2, 3, 3, 3, 3, 3, 4, 4” are obtained. In this case, the total value of the stress level values is divided by 10 to obtain a temporal average value for the 10 measurements, and is further divided by 4 to obtain an average value of the stress level values. In this example, (1+1+2+2+3+3+3+3+4+4)/10/4=0.65, so that “0.65” is the evaluation value indicating the abnormality level.

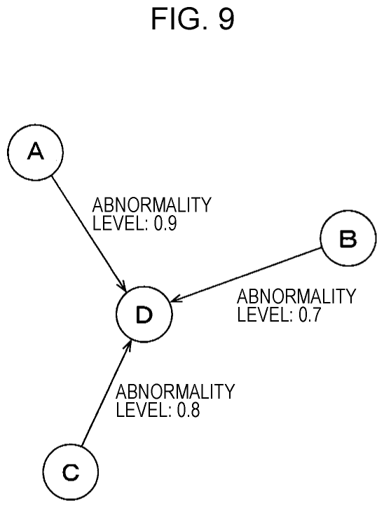

FIG. 9 illustrates an example of abnormality levels. The following description relates to an example where four people, namely, a user A, a user B, a user C, and a user D, are communicating with one another. As an example, these four people form a single organization (such as a team).

The second calculator 208 calculates that the abnormality level as a temporal average value of the stress level of the user A against the user D is 0.9, the abnormality level of the user B against the user D is 0.7, and the abnormality level of the user C against the user D is 0.8.

In this case, the determiner 204 determines whether or not any of these abnormality levels exceeds a predetermined threshold value (e.g., 0.5 for the sake of convenience). In the case of the example shown in FIG. 9, the abnormality levels of the user A, the user B, and the user C against the user D all exceed the predetermined threshold value. In such a case, the identifier 205 identifies that the user D applying the abnormality levels exceeding the predetermined threshold value to the multiple users is a person acting as the factor applying stress on the organization. This person (i.e., the user D) is an example of a person applying multiple abnormality levels that are higher than or equal to a predetermined value.

### Fourth Exemplary Embodiment

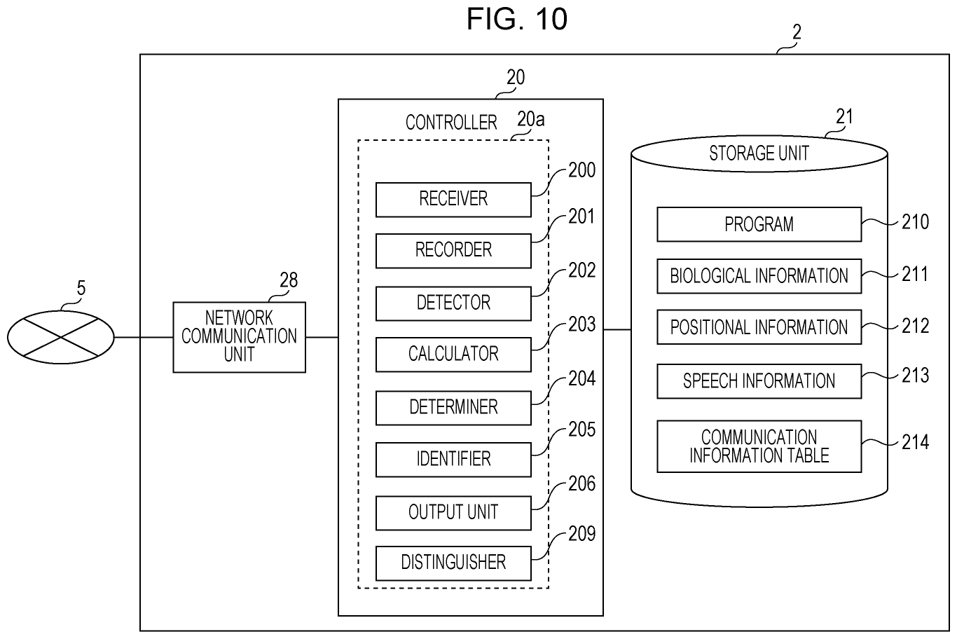

FIG. 10 is a block diagram illustrating an example of the configuration of an information processing apparatus 2 according to a fourth exemplary embodiment of the present disclosure. The controller 20 in the information processing apparatus 2 according to the second exemplary embodiment is different from that in the information processing apparatus 2 according to the first exemplary embodiment in that the controller 20 further includes a distinguisher 209. Components having configurations and functions identical to those in the first exemplary embodiment are given the same reference signs, and detailed descriptions thereof will be omitted. Moreover, the following description focuses on differences from the information processing apparatus 2 according to the first exemplary embodiment.

The processor 20a further functions as the distinguisher 209 by operating in accordance with the program 210 stored in the storage unit 21. The distinguisher 209 distinguishes the factor of stress from multiple factors. In detail, the distinguisher 209 distinguishes whether the factor of stress is work or a person.

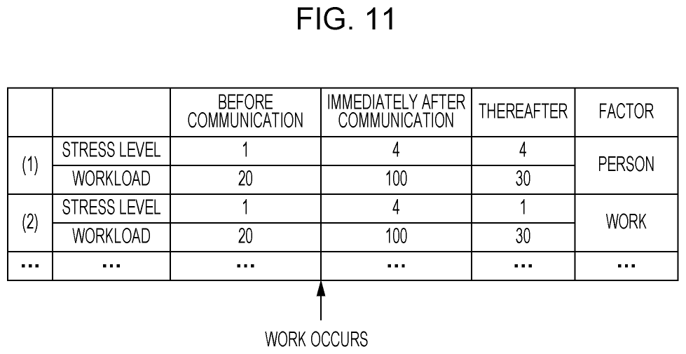

FIG. 11 illustrates an example of how the factor of stress is distinguished. Specifically, FIG. 11 is a table showing an example of variations in the stress level and the workload of a target user, and having a record of the stress level and the workload before communication, immediately after the communication, and thereafter (i.e., after a predetermined time period).

Similar to the first exemplary embodiment, the stress level is expressed with the integers “1”, “2”, “3”, and “4”. With regard to the workload, the value thereof increases with increasing amount of work such that a state where there is no work is “0” and a state where there is a large amount of work is “100”. The following description with reference to FIG. 11 relates to an example where a certain type of work occurs for a target user between “before communication” and “immediately after communication” (see arrow). The expression “work occurs” corresponds to a case where, for example, a new job is assigned to the target user from another user.

Case 1

For example, it is assumed that work occurs between the state before communication and the state immediately after the communication, and the workload increases from “20” to “100”, thus causing the stress level to increase from “1” to “4”. If the stress level remains at “4”, that is, at a high value, regardless of the work being subsequently completed such that the workload has decreased from “100” to “30” (see (1) in FIG. 11), the identifier 205 identifies that the factor applying stress on this target user is a “person”. In detail, the identifier 205 identifies that the factor applying stress on the target user is the person who has given the work thereto.

Case 2

Similar to Case 1, it is assumed that work occurs between the state before communication and the state immediately after the communication, and the workload increases from “20” to “100”, thus causing the stress level to increase from “1” to “4”. If the stress level decreases from “4” to “1” after the work is completed such that the workload decreases from “100” to “30” (see (2) in FIG. 11), the identifier 205 identifies that the factor applying stress on this target user is the “work”. In detail, the identifier 205 identifies that the factor applying stress on the target user is the relevant content of the work.

Although the exemplary embodiments of the present disclosure have been described above, the exemplary embodiments of the present disclosure are not limited to the exemplary embodiments described above, and various modifications are permissible so long as they do not depart from the scope of the disclosure. For example, in order to determine which users are communicating with each other, schedule information indicating the schedule of each user may be used in place of the positional information 212. With the schedule information, for example, users participating in the same meeting are identifiable.

Each component of the controller 20 may partially or entirely be constituted of a hardware circuit, such as a Field Programmable Gate Array (FPGA) or an Application Integrated Circuit (ASIC).

Furthermore, one or some of the components in each of the exemplary embodiments described above may be omitted or changed. Moreover, in the flowchart in each of the exemplary embodiments described above, for example, a step or steps may be added, deleted, changed, or interchanged within the scope of the disclosure. The program used in each of the exemplary embodiments described above may be provided by being recorded on a computer readable recording medium, such as a compact disc read-only memory (CD-ROM). Alternatively, the program used in each of the exemplary embodiments described above may be stored in an external server, such as a cloud server, and may be used via a network.

In the exemplary embodiments above, the term “processor” refers to hardware in a broad sense. Examples of the processor include general processors (e.g., CPU: Central Processing Unit) and dedicated processors (e.g., GPU: Graphics Processing Unit, ASIC: Application Integrated Circuit, FPGA: Field Programmable Gate Array, and programmable logic device).

In the exemplary embodiments above, the term “processor” is broad enough to encompass one processor or plural processors in collaboration which are located physically apart from each other but may work cooperatively. The order of operations of the processor is not limited to one described in the exemplary embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to
determine a level of load on a user in accordance with communication-related information of the user and biologically-related information of the user,
identify a factor that applies the load on the user in accordance with the level, and
output the identified factor.

2. The information processing apparatus according to claim 1,
wherein the processor performs control to identify the factor by using speech information and positional information as the communication-related information and to output the factor, the speech information indicating whether or not a speech is output by the user, the positional information indicating a position of the user.

3. The information processing apparatus according to claim 2,
wherein the processor performs the control to identify and output the factor in accordance with how the level of the load changes before and after the speech by the user.

4. The information processing apparatus according to claim 1,
wherein the processor performs control to identify that a person applying the load on the user is the factor and to output the factor in accordance with at least one evaluation value obtained by evaluating the level of the load in a time-series fashion.

5. The information processing apparatus according to claim 2,
wherein the processor performs the control to identify that a person applying the load on the user is the factor and to output the factor in accordance with at least one evaluation value obtained by evaluating the level of the load in a time-series fashion.

6. The information processing apparatus according to claim 3,
wherein the processor performs the control to identify that a person applying the load on the user is the factor and to output the factor in accordance with at least one evaluation value obtained by evaluating the level of the load in a time-series fashion.

7. The information processing apparatus according to claim 4,
wherein the at least one evaluation value comprises a plurality of evaluation values, and the user is one of a plurality of users, and
wherein, if the plurality of evaluation values obtained from the plurality of users are higher than or equal to a predetermined value, the processor performs the control to identify that a person applying the plurality of evaluation values higher than or equal to the predetermined value to the plurality of users is the factor and to output the factor.

8. The information processing apparatus according to claim 5,
wherein the at least one evaluation value comprises a plurality of evaluation values, and the user is one of a plurality of users, and
wherein, if the plurality of evaluation values obtained from the plurality of users are higher than or equal to a predetermined value, the processor performs the control to identify that a person applying the plurality of evaluation values higher than or equal to the predetermined value to the plurality of users is the factor and to output the factor.

9. The information processing apparatus according to claim 6,
wherein the at least one evaluation value comprises a plurality of evaluation values, and the user is one of a plurality of users, and
wherein, if the plurality of evaluation values obtained from the plurality of users are higher than or equal to a predetermined value, the processor performs the control to identify that a person applying the plurality of evaluation values higher than or equal to the predetermined value to the plurality of users is the factor and to output the factor.

10. A non-transitory computer readable medium storing a program causing a computer to execute a process, the process comprising:
determining a level of load on a user in accordance with communication-related information of the user and biologically-related information of the user;
identifying a factor that applies the load on the user in accordance with the level; and
outputting the identified factor.

11. An information processing apparatus comprising:
processing means for determining a level of load on a user in accordance with communication-related information of the user and biologically-related information of the user, identifying a factor that applies the load on the user in accordance with the level, and outputting the identified factor.
